(19) United States
(12) Patent Application Publication
(10) Pub. No.: US 2025/0206737 A1
(43) Pub. Date: Jun. 26, 2025

(54) PROCESS FOR PREPARING TUCATINIB AND ITS HEMIETHANOLATE

(71) Applicant: ScinoPharm Taiwan, Ltd., Tainan (TW)

(72) Inventors: Kuan-Hsun HUANG, Tainan (TW); Jing-Kai HUANG, Tainan (TW); Wen-Li SHIH, Tainan (TW); Jiunn-Cheh GUO, Tainan (TW); Tsung-Cheng HU, Tianan (TW)

(21) Appl. No.: 18/989,327

(22) Filed: Dec. 20, 2024

Related U.S. Application Data
(60) Provisional application No. 63/614,832, filed on Dec. 26, 2023.

(51) Int. Cl. C07D 471/04

(52) U.S. Cl. CPC C07D 471/04

(57) ABSTRACT

The present invention provides an improved process for the preparation of tucatinib, tucatinib intermediates, and the crystalline forms thereof. Specifically, the presently disclosed one-pot reaction processes save time and resources. The present invention improves the efficacy of producing Tucatinib, which can be performed under green chemistry.

### CROSS-REFERENCES TO RELATED APPLICATIONS

This application claims priority to U.S. Provisional Application No. 63/614,832, filed Dec. 26, 2023, which is incorporated herein in its entirety for all purposes.

### BACKGROUND OF THE INVENTION

Tucatinib (trade name Tukysa®) developed by Array BioPharma is a drug for the treatment of HER2-positive breast cancer. The chemical name of Tucatinib is N4-(4-([1,2,4]triazolo[1,5-a]pyridin-7-yloxy)-3-methylphenyl)-N6-(4,4-dimethyl-4,5-dihydrooxazol-2-yl)quinazoline-4,6-diamine.

On Apr. 17, 2020, the Food and Drug Administration (FDA) approved TUKYSA in dosage form of 50 mg and 150 mg oral tablets indicated for treating adult patients with advanced unresectable or metastatic HER2-positive breast cancer, including patients with brain metastases who have received one or more prior anti-HER2-based regimens in the metastatic setting by using Tucatinib in combination with trastuzumab and capecitabine.

The most common adverse reactions (>20% of patients) were diarrhea, palmar-plantar erythrodysesthesia, nausea, fatigue, hepatotoxicity, vomiting, stomatitis, decreased appetite, abdominal pain, headache, anemia, and rash. Tucatinib can also cause severe diarrhea and hepatotoxicity.

The recommended tucatinib dose is 300 mg taken orally twice a day in combination with trastuzumab (at standard dosage) and capecitabine (1000 mg/m2 given orally twice daily on days 1-14 of a 21-day cycle) until disease progression or unacceptable toxicity.

U.S. Pat. No. 8,648,087B2 reported the synthetic routes of Tucatinib on the milligram scale, as shown in Scheme 1. Later, improved synthetic route to Tucatinib were summarized in Scheme 2 to Scheme 4.

The process currently disclosed by Array BioPharma is shown in Scheme 1. The synthetic route starts from picolinic acid and aniline (Compound 5). Picolinic acid was converted to triazole (Compound 1) (5 steps with 24% yield) then reduced nitrobenzene to aniline (Compound 2). Thiourea (Compound 3) was synthesized from aniline (Compound 5) by three steps. 4-phenylaminequinazolin (Compound 4) was achieved by cyclization reaction between Compound 3 and Compound 2. Finally, Compound 4 was treated with NaOH and p-TsCl to give Tucatinib. This route involves multi-steps, and the purification process is basically completed by column chromatography. Also, it is prepared at the milligram scale in the laboratory to provide the API required for pharmaceutical research. There is no further disclosed that it can achieve large-scale preparation by Array BioPharma. In our study, yield of nitrobenzene (Compound 6) to thiourea (Compound 3) is 50% in two steps, althought the yield was improved 61% than Array Biopharma Inc (31% in two steps), but the yield is too low to apply to manufacture.

CN Pat. No. 109,942,576B reported the synthetic route of Tucatinib in a gram scale as Scheme 2. The route depicted delivered Tucatinib in 19% overall yield over eight steps from compound 8. A 100-gram scale of intermediate can be prepared. All intermediates were solids and can be purified by recrystallization. In the final coupling step, compound 19 is an unstable material, which would decompose after storing at room temperature for one week, and should be stored at −18° C. under argon protection. Also, Tucatinib was achieved by high reaction temperature (125° C.) in the final step and releases noxious methanethiol.

CN115,340,541A reported the process for preparing the thiourea intermediate (Compound 1), which is shown in Scheme 3. Aniline (Compound 2) was treated with formic acid to form quinazolinone (Compound 3). The compound 3 performed choloration by using SOCl₂ as solvent to give chloride of compound 4, the compound 4 could be subjected to the condensation reaction with aniline (compound 5) to form nitro quinazolinone (compound 6). The nitro group of the compound 6 is then reduced to afford compound 7 by Ni as a catalyst. The tucatinib precursor intermediate of compound 1 could be achieved by coupling aminoalcohol with TCDI. Since manufacture by using SOCl₂ as a solvent may cause corrosion of equipment and also not environmentally friendly.

The literature, Synthetic Route of Tucatinib in Organic Preparations and Procedures International 2021, 53(6), 554-56, disclosed one of the synthetic route for the Tucatinib as shown in Scheme 4. The yield of the key intermediate of compound 5 prepared from compound 1 and compound 4 in four steps is 46%. Subsequently, ring closing and catalytic hydrogenation reaction were achieved aniline (compound 8) with 82% yield over 2 steps. The aniline (compound 8) was reacted with aminoalcohol and TCDI to give thiourea (compound 9) in 81% and further cyclization by TsCl and NaOH to give Tucatinib with 77% yield.

Despite the above-described processes, there remains a need for the development of more efficient and improved processes for the preparation of Tucatinib. The present disclosure addresses this need and provides related advantages as well.

### SUMMARY OF THE INVENTION

The first aspect of the present invention is a crystalline form of Formula M3 (hereafter designated as crystalline Form A of Formula M3) characterized by a powder X-ray diffraction (“PXRD”) pattern with peaks at about 11.3, 11.5, 13.7, 21.5 and 26.0±0.2 degrees two-theta.

The crystalline Form A of Formula M3 is preferably characterized by a powder X-ray diffraction pattern as substantially depicted in FIG. 1.

The crystalline Form A of Formula M3 may be prepared by dissolving Formula M2 in NMP and adding 10% Pd/C to form a reaction mixture; stirring the reaction mixture under pressure of H₂ at 50° C. The resulting mixture is filtered to remove Pd/C, washed with NMP, and subsequently H₂O is added to the resulting filtrate to precipitate M3. The slurry is cooled to 5° C. and filtered. The wet cake was dried at 60° C. to obtain the crystalline Form A of Formula M3

The second aspect of the present invention is a crystalline form of Formula M3 (hereafter designated as crystalline Form B of Formula M3) characterized by a powder X-ray diffraction (“PXRD”) pattern with peaks at about 11.9, 13.1, 15.9, 20.7, 24.2±0.2 degrees two-theta.

The crystalline Form B of Formula M3 is preferably characterized by a powder X-ray diffraction pattern as substantially depicted in FIG. 2.

The crystalline Form B of Formula M3 may be prepared by dissolving Formula M2 in NMP and adding 10% Pd/C to form a reaction mixture; stirring the reaction mixture under pressure of H₂ at 50° C. The resulting mixture is filtered to remove Pd/C and washed with NMP, and subsequently H₂O is added to the resulting filtrate to precipitate M3. The slurry is cooled to 5° C. and filtered. The wet cake was dried at 80° C. to obtain the crystalline Form B of Formula M3.

The third aspect of the present invention is a crystalline form of Formula M3 (hereafter designated as crystalline Form C of Formula M3) characterized by a powder X-ray diffraction (“PXRD”) pattern with peaks at about 10.8, 13.1, 17.2, 24.9, and 27.2±0.2 degrees two-theta.

The crystalline Form C of Formula M3 is preferably characterized by a powder X-ray diffraction pattern as substantially depicted in FIG. 3.

The crystalline Form C of Formula M3 may be prepared by dissolving Formula M2 in NMP and adding 10% Pd/C to form a reaction mixture; stirring the reaction mixture under pressure of H₂ at 50° C. The resulting mixture is filtered to remove Pd/C and washed with NMP, and subsequently ACN is added to the resulting filtrate to precipitate M3. The slurry is cooled to 5° C. and filtered. The wet cake was dried at 60° C. to obtain the crystalline Form C of Formula M3.

In addition, the present invention is also directed to processes for transforming Formula M3 to Tucatinib by one-pot reaction.

### BRIEF DESCRIPTION OF THE DRAWINGS

FIG. 1 illustrates a powder X-ray diffraction pattern of crystalline Form A Tucatinib Formula M3 characterized by a powder X-ray diffraction pattern.

FIG. 2 illustrates a powder X-ray diffraction pattern of crystalline Form B Tucatinib Formula M3 characterized by a powder X-ray diffraction pattern.

FIG. 3 illustrates a powder X-ray diffraction pattern of crystalline Form C Tucatinib Formula M3 characterized by a powder X-ray diffraction pattern.

FIG. 4 illustrates Scheme 1, the synthetic route disclosed in U.S. Pat. No. 8,648,087B2.

FIG. 5 illustrates Scheme 2, the synthetic route disclosed in CN 109,942,576B.

FIG. 6 illustrates Scheme 3, the synthetic route disclosed in CN115,340,541A.

FIG. 7 illustrates Scheme 4, the synthetic route disclosed in Organic Preparations and Procedures International 2021, 53(6), 554-561.

### DETAILED DESCRIPTION OF THE INVENTION

### I. General

The present invention is directed to processes of making crystalline forms of Tucatinib intermediate Formula M3, designated herein as Form A, Form B, and Form C of Formula M3, and using Formula M3 to prepare Tucatinib by one-pot reaction. Further, this invention also discloses the method of preparing Tucatinib hemiethanolate.

### II. Definitions

As used herein, the term “solvent” refers to a substance that is liquid at ambient temperature and pressure, capable of dissolving a solute. Examples of solvents include water, and organic solvents such as water, formamide (DMF), dimethyl acetamide (DMAc), tetrahydrofuran (THF), dimethyl sulfoxide (DMSO), N-methylpyrrolidone (NMP), methylene chloride (DCM), methanol (MeOH), and ethanol (EtOH). Mixtures of solvents include combinations of two, three, four or more, of the noted solvents.

As used herein, the term “anti-solvent” refers to a solvent which is less capable or incapable of dissolving a solute. Examples of anti-solvents include water, ethanol (EtOH), acetonitrile (ACN), toluene, ethyl acetate (EtOAc), acetone, hexane, heptane. Mixtures of anti-solvents include combinations of two, three, four or more, of the solvents or anti-solvents.

As used herein, the terms “reacting” refers to the process of bringing into contact at least two distinct species such that they can react. It should be appreciated, however, that the resulting reaction product can be produced directly from a reaction between the added reagents or from an intermediate from one or more of the added reagents that can be produced in the reaction mixture.

As used herein, the term “cyclizing” or “cyclization reaction” refers to the process of forming a cyclic structure or a ring compound from an acyclic precursor molecule by the creation of a new bond. Representative cyclizing reagents for use in the cyclization reaction include, but are not limited to, air, mCPBA, tert-Butyl Hydroperoxide, oxane, O₂, O₃, or H₂O₂.

As used herein, the term “hydrogenating” refers to the process of reducing a functional group, such as reducing a nitro group (−NO₂) to an amine (−NH₂). Representative hydrogenating agents include, but are not limited to H₂ with a suitable catalyst such as Pd/C.

As used herein, the term “one-pot process” refers to a process involving two or more chemical transformations that occur in a single reaction vessel.

As used herein, the term “coupling reagent” refers to a reagent useful for coupling two distinct chemical groups. Representative coupling reagents include, but are not limited to, 1,1-thiocarbonyldiimidazole (TCDI), thiophosgene, 1,1′-Thiocarbonyldi-2(1H)-pyridone or Di-2-pyridyl thionocarbonate.

### III. Methods of Making Tucatinib

In some embodiments, the present invention provides a process for preparing a crystalline polymorph of Formula M3

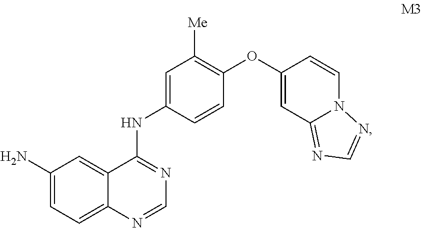 [SMILES: Cc1cc(Nc2ncnc3ccc(N)cc23)ccc1Oc1ccn2ncnc2c1]

comprising:
a) hydrogenating N-[3-Methyl-4-([1,2,4]triazolo[1,5-a]pyridin-7-yloxy)phenyl]-6-nitro-4-quinazolinamine of Formula M2:

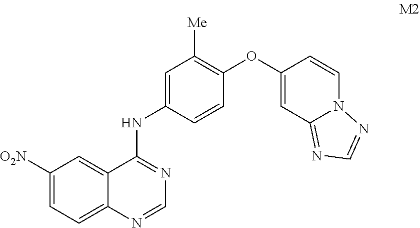 [SMILES: Cc1cc(Nc2ncnc3ccc([N+](=O)[O-])cc23)ccc1Oc1ccn2ncnc2c1]

in a suitable solvent to provide Formula M3;
b) adding an suitable anti-solvent to the solution of step (a);
c) recovering Formula M3 as a crystalline form; and
d) optionally purifying the crystallized Formula M3.

In some embodiments, the present invention provides the process of preparing the crystalline polymorph of Formula M3, wherein the suitable solvent is DMF, DMAc, THF, DMSO, NMP, DCM, MeOH, H₂O, or a combination thereof. In some embodiments, the present invention provides the process of preparing the crystalline polymorph of Formula M3, wherein the suitable solvent is NMP. In some embodiments, the present invention provides the process of preparing the crystalline polymorph of Formula M3, wherein the suitable solvent is THF and H₂O.

In some embodiments, the present invention provides the process of preparing the crystalline polymorph of Formula M3, wherein the suitable anti-solvent is ACN, H₂O, toluene, EtOAc, acetone, hexane, heptane, or a combination thereof. In some embodiments, the present invention provides the process of preparing the crystalline polymorph of Formula M3, wherein the suitable anti-solvent is ACN.

In some embodiments, the present invention provides the process of preparing the crystalline polymorph of Formula M3, comprising
a) hydrogenating Formula M2 with 10% Pd/C and H₂ in NMP to provide Formula M3; and
b) recovering Formula M3 as a crystalline form.

In some embodiments, the present invention provides the process of preparing the crystalline polymorph of Formula M3, comprising
a) hydrogenating Formula M2 with 10% Pd/C and H₂ in NMP to provide Formula M3;
b) adding ACN to the solution of step (a); and
c) recovering Formula M3 as a crystalline form.

In some embodiments, the present invention provides the process of preparing the crystalline polymorph of Formula M3, comprising
a) hydrogenating Formula M2 with 10% Pd/C and H₂ in THF and H₂O to provide Formula M3; and
b) recovering Formula M3 as a crystalline form.

In some embodiments, the present invention provides the process of preparing the crystalline polymorph of Formula M3, comprising
a) hydrogenating Formula M2 with 10% Pd/C and H₂ in THF and H₂O to provide Formula M3;
b) adding ACN to the solution of step (a); and
c) recovering Formula M3 as a crystalline form.

In some embodiments, the present invention provides an isolated crystalline form of N⁴-(3-methyl-4-[[1,2,4]triazolo[1,5-a]pyridin-7-yloxy]phenyl)quinazoline-4,6-diamine of Formula M3:

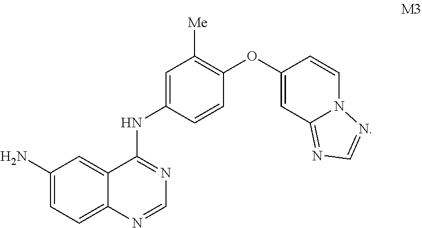 [SMILES: Cc1cc(Nc2ncnc3ccc(N)cc23)ccc1Oc1ccn2ncnc2c1]

In some embodiments, the present invention provides an isolated crystalline form of N⁴-(3-methyl-4-[[1,2,4]triazolo[1,5-a]pyridin-7-yloxy]phenyl)quinazoline-4,6-diamine of Formula M3, wherein the polymorph is Form A with X-Ray Diffraction Pattern (XRPD) peaks at 11.3, 11.5, 13.7, 21.5 and 26.0±0.2 degrees 2θ (CuKα).

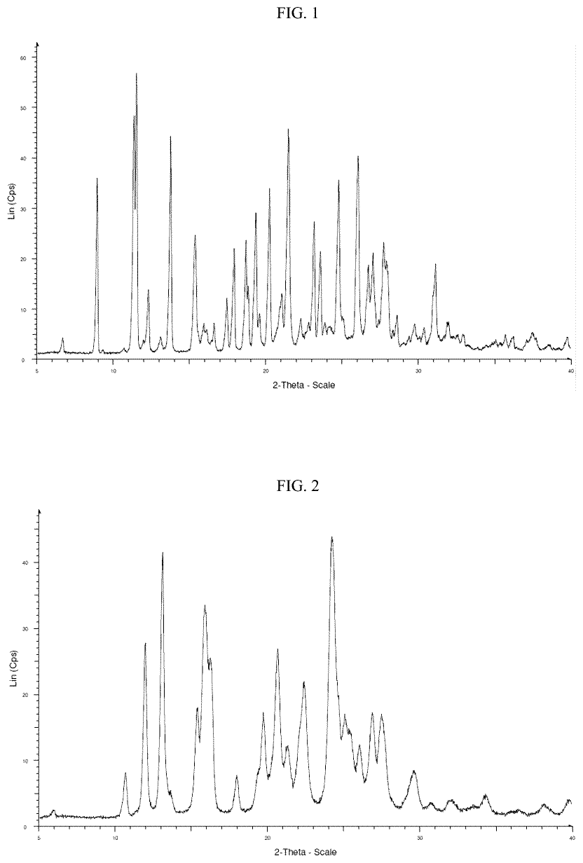

In some embodiments, the present invention provides the isolated crystalline form of N⁴-(3-methyl-4-[[1,2,4]triazolo[1,5-a]pyridin-7-yloxy]phenyl)quinazoline-4,6-diamine of Formula M3, wherein the polymorph Form A is hygroscopic. In some embodiments, the present invention provides the isolated crystalline form of N⁴-(3-methyl-4-[[1,2,4]triazolo[1,5-a]pyridin-7-yloxy]phenyl)quinazoline-4,6-diamine of Formula M3, wherein the polymorph Form A has an XRPD pattern substantially the same as FIG. 1.

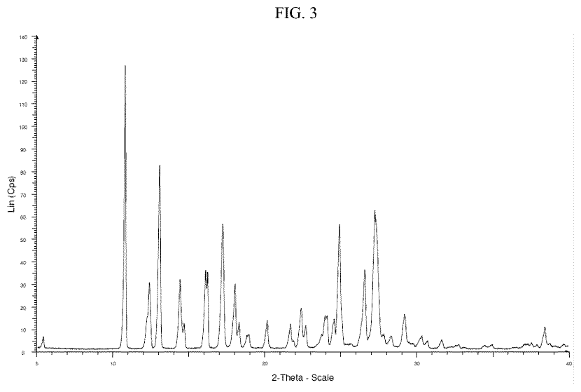

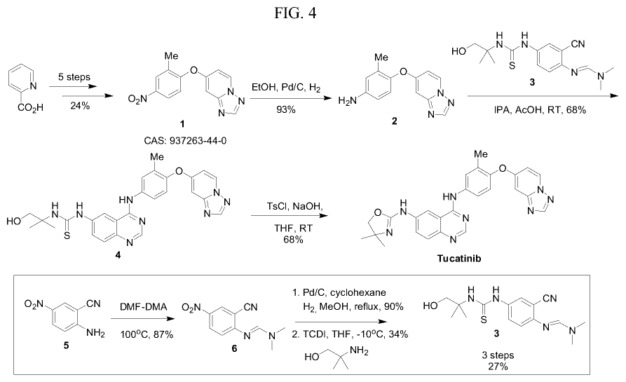

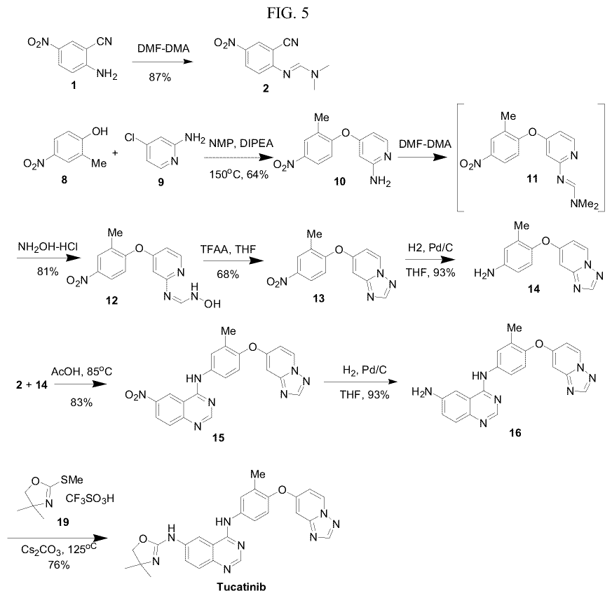

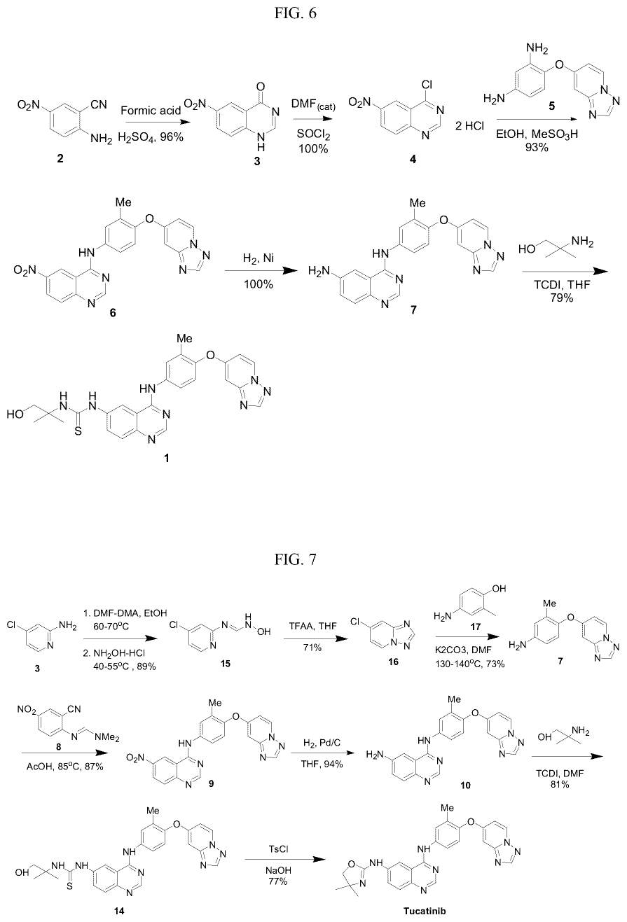

In some embodiments, the present invention provides the isolated crystalline form of N⁴-(3-methyl-4-[[1,2,4]triazolo[1,5-a]pyridin-7-yloxy]phenyl)quinazoline-4,6-diamine of Formula M3, wherein the polymorph is Form B with X-Ray Diffraction Pattern (XRPD) peaks at 11.9, 13.1, 15.9, 20.7, and 24.2±0.2 degrees 2θ (CuKα). In some embodiments, the present invention provides the an isolated crystalline form of N⁴-(3-methyl-4-[[1,2,4]triazolo[1,5-a]pyridin-7-yloxy]phenyl)quinazoline-4,6-diamine of Formula M3, wherein the polymorph Form B is non-hygroscopic. In some embodiments, the present invention provides a the n isolated crystalline form of N⁴-(3-methyl-4-[[1,2,4]triazolo[1,5-a]pyridin-7-yloxy]phenyl)quinazoline-4,6-diamine of Formula M3, wherein the polymorph Form B has an XRPD pattern substantially the same as FIG. 2.

In some embodiments, the present invention provides a the n isolated crystalline form of N⁴-(3-methyl-4-[[1,2,4]triazolo[1,5-a]pyridin-7-yloxy]phenyl)quinazoline-4,6-diamine of Formula M3, wherein the polymorph is Form C with X-Ray Diffraction Pattern (XRPD) peaks at 10.8, 13.1, 17.2, 24.9, and 27.2±0.2 degrees 2θ (CuKα). In some embodiments, the present invention provides an isolated crystalline form of N⁴-(3-methyl-4-[[1,2,4]triazolo[1,5-a]pyridin-7-yloxy]phenyl)quinazoline-4,6-diamine of Formula M3, wherein the polymorph Form C is non-hygroscopic. In some embodiments, the present invention provides an isolated crystalline form of N⁴-(3-methyl-4-[[1,2,4]triazolo[1,5-a]pyridin-7-yloxy]phenyl)quinazoline-4,6-diamine of Formula M3, wherein the polymorph has an XRPD pattern substantially the same as FIG. 3.

In some embodiments, the present invention provides a one-pot process for preparing the compound of Formula (I):

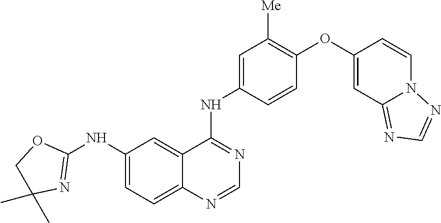 [SMILES: Cc1cc(Nc2ncnc3ccc(NC4=NC(C)(C)CO4)cc23)ccc1Oc1ccn2ncnc2c1]

or a pharmaceutically acceptable salt thereof, the process comprising:
a) reacting Formula M3:

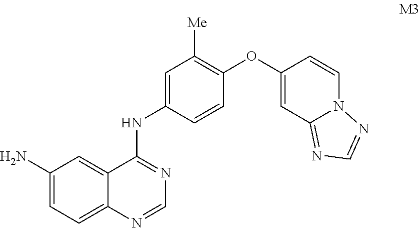 [SMILES: Cc1cc(Nc2ncnc3ccc(N)cc23)ccc1Oc1ccn2ncnc2c1]

with a coupling reagent and Formula SM3:

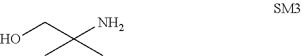

in a suitable solvent system to provide M4 reaction mixture,

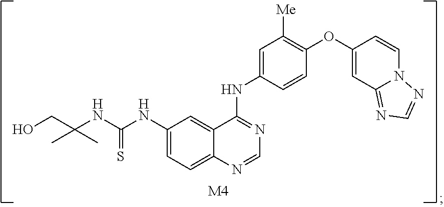

b) converting the M4 reaction mixture to Formula (I); and
c) optionally purify the Formula I.

In some embodiments, the present invention provides a one-pot process for preparing the compound of Formula (I), wherein the coupling reagent is 1,1-thiocarbonyldiimidazole (TCDI), thiophosgene, 1,1′-Thiocarbonyldi-2(1H)-pyridone or Di-2-pyridyl thionocarbonate. In some embodiments, the present invention provides the one-pot process for preparing the compound of Formula (I), wherein the coupling reagent is TCDI.

In some embodiments, the present invention provides the one-pot process for preparing the compound of Formula (I), wherein the suitable solvent system is DMF, DMAc, DMSO, NMP. THF, or DCM. In some embodiments, the present invention provides the one-pot process for preparing the compound of Formula (I), wherein the suitable solvent system is DMF.

In some embodiments, the present invention provides a one-pot process for preparing the compound of Formula (I), wherein the step a) comprises reacting Formula M3 and the coupling reagent TCDI in DMF, to produce a M3IM reaction mixture:

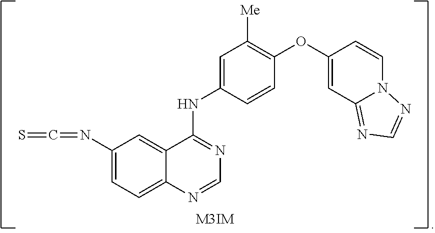

In some embodiments, the present invention provides the one-pot process for preparing the compound of Formula (I), wherein the step a) further comprises combining the M3IM reaction mixture and Formula SM3:

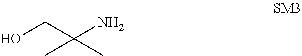

to produce the M4 reaction mixture.

In some embodiments, the present invention provides the one-pot process for preparing the compound of Formula (I), wherein the step (b) is performed by cyclization reaction. In some embodiments, the present invention provides the one-pot process for preparing the compound of Formula (I), wherein the step (b) further comprises air, mCPBA, tert-Butyl Hydroperoxide, oxane, O₂, O₃, or H₂O₂ to convert the M4 reaction mixture to Formula (I). In some embodiments, the present invention provides the one-pot process for preparing the compound of Formula (I), wherein the step (b) further comprises air to convert the M4 reaction mixture to Formula (I).

In some embodiments, the present invention provides the one-pot process for preparing the compound of Formula (I), wherein the Formula M3 is an isolated crystalline form of Formula M3.

In some embodiments, the present invention provides the one-pot process for preparing the compound of Formula (I), the process comprising
a) reacting Formula M3 with TCDI and Formula SM3 in DMF to provide the M4 reaction mixture; and
b) cyclizing the M4 reaction mixture to Formula (I) with air.

In some embodiments, the present invention provides a process of preparing a compound of Formula (I)

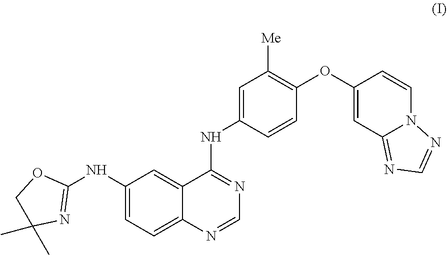 [SMILES: Cc1cc(Nc2ncnc3ccc(NC4=NC(C)(C)CO4)cc23)ccc1Oc1ccn2ncnc2c1]

or a pharmaceutically acceptable salt thereof, the process comprising:
adding a suitable cyclizing reagent to a M4 reaction mixture:

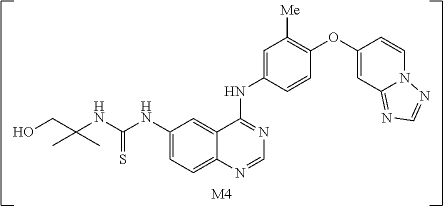

to prepare the compound of Formula I.

In some embodiments, the present invention provides the process of preparing a compound of Formula (I), wherein the suitable cyclizing reagent comprises: air, mCPBA, tert-Butyl Hydroperoxide, oxane, O₂, O₃, or H₂O₂. In some embodiments, the present invention provides the process of preparing a compound of Formula (I), wherein the cyclizing reagent comprises air.

In some embodiments, the present invention provides the process of preparing a compound of Formula (I), wherein the M4 reaction mixture is prepared by reacting Formula M3:

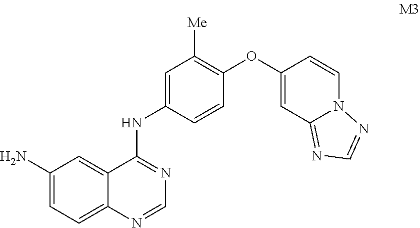 [SMILES: Cc1cc(Nc2ncnc3ccc(N)cc23)ccc1Oc1ccn2ncnc2c1]

with TCDI and Formula SM3:

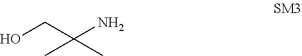

in DMF to provide the M4 reaction mixture.

In some embodiments, the present invention provides a process of preparing a compound of Formula (I), wherein the compound of Formula I is prepared by reacting Formula M3:

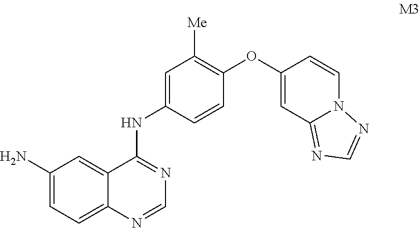 [SMILES: Cc1cc(Nc2ncnc3ccc(N)cc23)ccc1Oc1ccn2ncnc2c1]

with TCDI and Formula SM3:

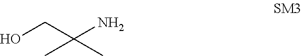

in DMF to provide the M4 reaction mixture; and
adding air to the M4 reaction mixture to prepare the compound of Formula I.

In some embodiments, the present invention provides a process of preparing the compound of Formula (I) or a pharmaceutically acceptable salt thereof, the process comprising:
using a crystallized form of Formula M3 of any one of claims 7 to 16 to perform a one-pot reaction of any one of claims 17 to 28.

In some embodiments, the present invention provides a process for preparing Tucatinib hemiethanolate, comprising dissolving Tucatinib API in DMSO and EtOH to prepare Tucatinib hemiethanolate.

In some embodiments, the present invention provides a process for preparing Tucatinib hemiethanolate, comprising dissolving Tucatinib API in a suitable organic solvent or a mixture of organic solvents and adding EtOH to prepare Tucatinib hemiethanolate. In some embodiments, the process of preparing Tucatinib hemiethanolate includes the process wherein the suitable organic solvent is DMSO.

### IV. Examples

The following examples are provided to illustrate, but not to limit, the present invention. Examples described herein comprise a process for making crystalline forms of Formula M3 and using Formula M3 to prepare Tucatinib by one-pot reaction for either laboratory-scale or industrial scale.

The X-ray powder diffraction (XRPD) patterns provided herein of the solid forms of N⁴-(4-([1,2,4]triazolo[1,5-a]pyridin-7-yloxy)-3-methylphenyl)quinazoline-4,6-diamine (Compound M3) were collected using Cu Kα radiation.

### Example 1. The Preparation of the Formula M2

Formula SM1 (53.56 g, 328.33 mmole) and N,N-Dimethylformamide dimethyl acetal (40.27 g, 337.98 mmole) in Toluene (643 mL) were heated to 70° C., after 2 hrs, the resulting mixture was cooled to 60° C. followed by adding Formula SM2 (77.34 g, 321.89 mmole) and AcOH (302 mL). The Formula M2 was solid out by addition water (178 mL). The mixture was cooled to RT, filtered and washed with toluene (160 mL) and water (270 mL). The wet cake was dried to give M2 (126.3 g) with isolated in 91% yield.

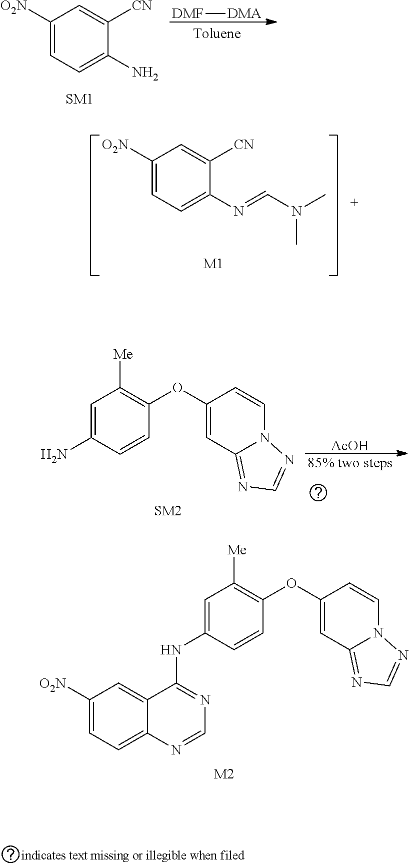

### Example 2: The Preparation of the Hygroscopic Form of Formula M3

(a) The Formula M2 (15 g, 36.28 mmol) in NMP (105 mL) is added 10% Pd/C (0.45 g). The reaction mixture is stirred under pressure of H₂ at 50° C. The resulting mixture is filtered through celite bed to remove Pd/C and washed with NMP (45 mL) then H₂O is added (165 mL) to the resulting filtrate to precipitate M3. The slurry is cooled to 5° C. and filtered. The wet cake is washed with H₂O (45 mL) and then dried at 60° C. to give M3 in 84% yield.

(b) The Formula M2 (15 g, 36.28 mmol) in THF/H₂O=7/3(v/v) (300 mL) is added 10% Pd/C (0.45 g). The reaction mixture is stirred under pressure of H₂ at 50° C. The resulting mixture is filtered through celite bed to remove Pd/C and washed with THF/H₂O=7/3(v/v) (75 mL) then H₂O is added (195 mL) to the resulting filtrate to precipitate M3. The slurry is cooled to 5° C. and filtered. The wet cake is washed with H₂O (45 mL) and then dried at 60° C. to give M3 in 95% yield.

(c) The PXRD pattern of the Form A of Formula M3 was measured and confirmed to have the pattern as illustrated in FIG. 1. The PXRD characteristics of the crystalline Form A of Formula M3 are reflected in the following table:

| Angle | Intensity | Intensity % |
|---|---|---|
| 2-Theta ° | Cps | % |
| 6.66 | 4.2 | 7.4 |
| 8.90 | 36 | 63.4 |
| 9.28 | 1.87 | 3.3 |
| 10.67 | 2.23 | 3.9 |
| 11.32 | 48.4 | 85.1 |
| 11.49 | 56.8 | 100 |
| 11.97 | 3.79 | 6.7 |
| 12.27 | 13.8 | 24.3 |
| 13.08 | 4.45 | 7.8 |
| 13.73 | 44.3 | 78 |
| 15.35 | 24.6 | 43.3 |
| 15.92 | 7.08 | 12.5 |
| 16.10 | 5.83 | 10.3 |
| 16.58 | 7.16 | 12.6 |
| 17.43 | 12.1 | 21.3 |
| 17.9 | 22 | 38.6 |
| 18.68 | 23.6 | 41.5 |
| 18.85 | 14.5 | 25.5 |
| 19.33 | 29.1 | 51.3 |
| 19.57 | 9.03 | 15.9 |
| 20.23 | 34 | 59.7 |
| 21.04 | 13 | 22.8 |
| 21.48 | 45.8 | 80.5 |
| 22.28 | 8.11 | 14.3 |
| 22.81 | 7.35 | 12.9 |
| 23.14 | 27.3 | 48 |
| 23.57 | 21.4 | 37.6 |
| 23.86 | 7.28 | 12.8 |
| 24.16 | 6.35 | 11.2 |
| 24.77 | 35.7 | 62.7 |
| 26.04 | 40.4 | 71.1 |
| 26.72 | 18.7 | 32.9 |
| 27.03 | 21.1 | 37.1 |
| 27.73 | 23.2 | 40.8 |
| 27.93 | 19.5 | 34.3 |
| 28.33 | 5.81 | 10.2 |
| 28.59 | 8.7 | 15.3 |
| 29.03 | 3.32 | 5.8 |
| 29.39 | 4.58 | 8.1 |
| 29.75 | 6.99 | 12.3 |
| 30.06 | 4.12 | 7.2 |
| 30.38 | 6.27 | 11 |
| 30.97 | 12.7 | 22.3 |
| 31.12 | 18.9 | 33.3 |
| 31.93 | 7.27 | 12.8 |
| 32.56 | 4.71 | 8.3 |
| 32.94 | 4.95 | 8.7 |
| 35.10 | 3.89 | 6.9 |
| 35.37 | 3.29 | 5.8 |
| 35.72 | 4.87 | 8.6 |
| 36.21 | 4.2 | 7.4 |
| 37.50 | 5.33 | 9.4 |
| 38.56 | 2.79 | 4.9 |
| 39.78 | 4.37 | 7.7 |

### Example 3: The Preparation of the Non-Hygroscopic Form of Formula M3

(a) The Formula M2 (15 g, 36.28 mmol) in NMP (105 mL) is added 10% Pd/C (0.45 g). The reaction mixture is stirred under pressure of H₂ at 50° C. The resulting mixture is filtered through celite bed to remove Pd/C and washed with NMP (45 mL) then H₂O is added (300 mL) to the resulting filtrate to precipitate M3. The slurry is cooled to 5° C. and filtered. The wet cake is washed with H₂O (45 mL) and then dried at 80° C. to give crystalline Form B of M3 in 88% yield.

(b) The PXRD pattern of the Form B of Formula M3 was measured and confirmed to have the pattern as illustrated in FIG. 2. The PXRD characteristics of the crystalline Form B of Formula M3 are reflected in the following table:

| Angle | Intensity | Intensity % |
|---|---|---|
| 2-Theta ° | Cps | % |
| 5.92 | 2.36 | 5.4 |
| 10.65 | 8.09 | 18.4 |
| 11.94 | 27.6 | 62.9 |
| 13.08 | 41.5 | 94.6 |
| 15.36 | 17.9 | 40.8 |
| 15.88 | 33.5 | 76.3 |
| 16.24 | 25.5 | 58 |
| 17.97 | 7.7 | 17.5 |
| 19.72 | 17.2 | 39.3 |
| 20.66 | 26.9 | 61.2 |
| 21.29 | 12.2 | 27.7 |
| 22.07 | 14.9 | 33.9 |
| 22.39 | 22 | 50 |
| 24.23 | 43.9 | 100 |
| 25.08 | 17 | 38.7 |
| 25.39 | 14.5 | 32.9 |
| 26.03 | 12.3 | 28.1 |
| 26.88 | 17.2 | 39.2 |
| 27.49 | 17 | 38.6 |
| 29.61 | 8.31 | 18.9 |
| 30.72 | 3.41 | 7.8 |
| 32.06 | 4 | 9.1 |
| 34.35 | 4.61 | 10.5 |
| 38.15 | 3.36 | 7.7 |
| 39.80 | 3.95 | 9 |

(c) The Formula M2 (25 g, 57.95 mmol) in NMP (175 mL) is added 10% Pd/C (0.75 g). The reaction mixture is stirred under pressure of H₂ at 50° C. The resulting mixture is filtered through celite bed to remove Pd/C and washed with NMP (75 mL) then ACN is added (450 mL) to the resulting filtrate to precipitated M3. The slurry is cooled to 5° C. and filtered. The wet cake is washed with ACN (75 mL) and then dried at 80° C. to provide M3 (19.34 g) was obtained in 86% yield.

(d) Formula M2 (15 g, 34.79 mmol) in THF/H₂O=7/3(v/v) (300 mL) is added 10% Pd/C (0.45 g). The reaction mixture is stirred under pressure of H₂ at 50° C. The resulting mixture is filtered through celite bed to remove Pd/C and washed with THF/H₂O=7/3(v/v) (75 mL) then ACN is added (150 mL) to the resulting filtrate to precipitated M3. The slurry is cooled to 5° C. and filtered. The wet cake is washed with ACN (75 mL) and then dried at 80° C. to provide M3 (12.66 g) was obtained in 95% yield.

(e) The PXRD pattern of the Form C of Formula M3 was measured and confirmed to have the pattern as illustrated in FIG. 3. The PXRD characteristics of the crystalline Form C of Formula M3 are reflected in the following table:

| Angle | Intensity | Intensity % |
|---|---|---|
| 2-Theta ° | Cps | % |
| 5.37 | 6.72 | 5.3 |
| 10.77 | 127 | 100 |
| 12.15 | 12.9 | 10.1 |
| 12.37 | 30.7 | 24.1 |
| 13.04 | 82.9 | 65.2 |
| 14.40 | 32 | 25.2 |
| 14.64 | 12.6 | 9.9 |
| 16.15 | 31.3 | 24.6 |
| 17.21 | 56.8 | 44.6 |
| 18.00 | 30.1 | 23.6 |
| 18.28 | 13 | 10.2 |
| 18.89 | 7.55 | 5.9 |
| 20.13 | 13.8 | 10.9 |
| 21.65 | 12.3 | 9.7 |
| 21.90 | 4.77 | 3.8 |
| 22.36 | 19.3 | 15.2 |
| 22.67 | 11.6 | 9.1 |
| 23.69 | 7.36 | 5.8 |
| 24.03 | 15 | 11.8 |
| 24.52 | 14.4 | 11.3 |
| 24.88 | 56.5 | 44.4 |
| 26.56 | 36.4 | 28.6 |
| 27.26 | 62.8 | 49.4 |
| 27.8 | 7.94 | 6.2 |
| 28.29 | 6.96 | 5.5 |
| 29.19 | 16.6 | 13 |
| 30.30 | 6.53 | 5.1 |
| 30.68 | 4.57 | 3.6 |
| 31.63 | 5.14 | 4 |
| 34.47 | 2.87 | 2.3 |
| 34.93 | 3.03 | 2.4 |
| 38.43 | 11 | 8.7 |

### Example 4: The Preparation Method of Using the Formula M3 to Prepare Tucatinib in One-Pot

The Formula M3 (20.49 g, 53.44 mmol) and TCDI (10.08 g, 1.03 equiv.) in DMF (140 mL) is stirred at NMT 10° C. The Formula SM3 (14.29 g) in DMF (20.5 mL) is added and rinsed with DMF (20.5 mL). A suitable cyclizing reagent or a mixture of cyclizing reagents, such as air, is added to the M4 reaction mixture to operate the cyclization reaction. After reaction is completed, ACN (510 mL) is added the reaction mixture to precipitate the Formula 1. Cool the mixture to 5° C. then filter and wash the mixture with ACN (100 mL) and PPW (100 mL). The wet cake is dried to give API (19.70 g) was isolated in 78.46%.

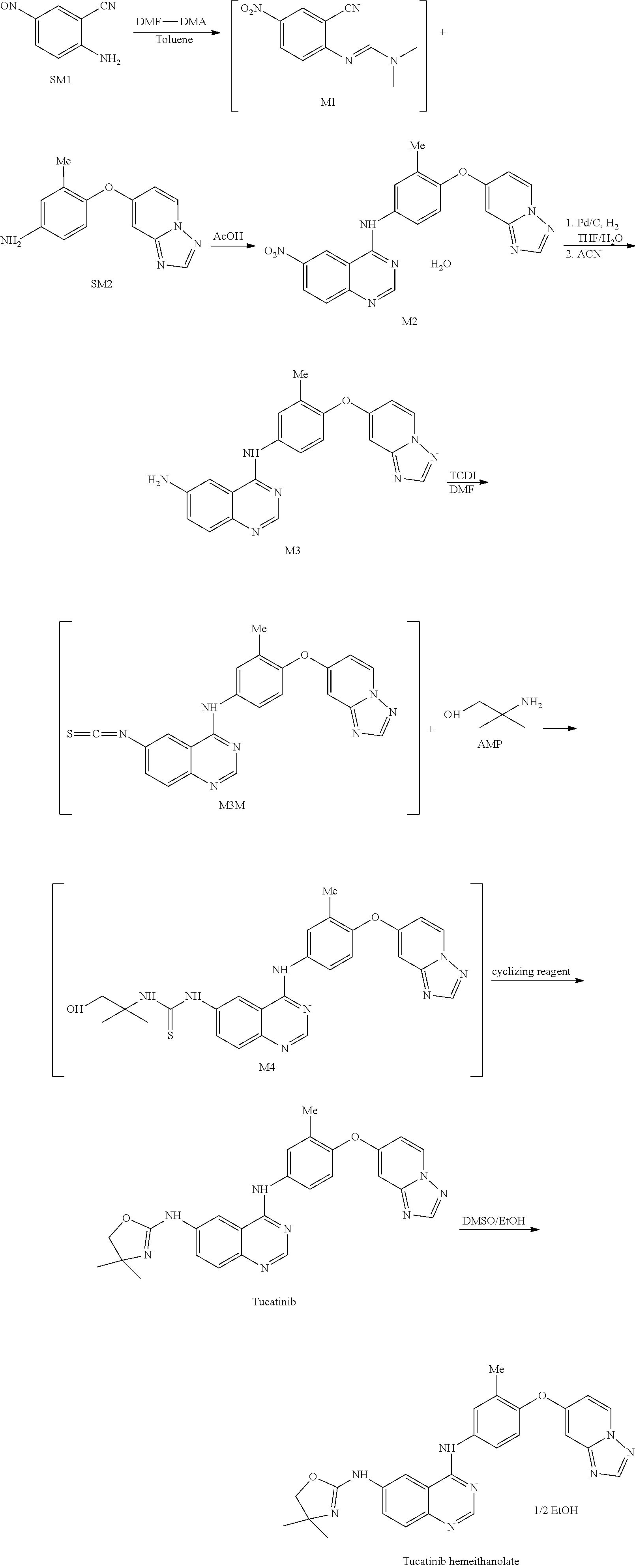

### Example 5: The Preparation of Tucatinib Hemiethanolate

Tucatinib (1.00 g) is dissolved in DMSO (3 mL) at 78° C. EtOH (5 mL) is added followed by adding the seed API hemiethanolate (10 mg) then rinse with EtOH (0.5 mL) and stirred for 2 hrs. The resulting slurry was stirred at RT and added EtOH (6 mL.). The slurry was stirred at 0-10° C. then filtered, washed with EtOH (3 mL, 3 vol.). The wet cake was dried to give API hemiethanolate (865 mg) with 81.76% yield.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A process for preparing a crystalline polymorph of Formula M3 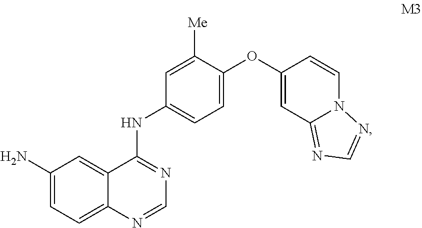 [SMILES: Cc1cc(Nc2ncnc3ccc(N)cc23)ccc1Oc1ccn2ncnc2c1]
comprising:
a) hydrogenating N-[3-Methyl-4-([1,2,4]triazolo[1,5-a]pyridin-7-yloxy)phenyl]-6-nitro-4-quinazolinamine of Formula M2: 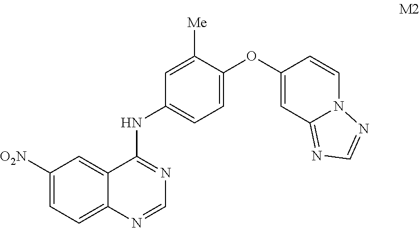 [SMILES: Cc1cc(Nc2ncnc3ccc([N+](=O)[O-])cc23)ccc1Oc1ccn2ncnc2c1]
in a suitable solvent to provide Formula M3;
b) adding an suitable anti-solvent to the solution of step (a);
c) recovering Formula M3 as a crystalline form; and
d) optionally purifying the crystallized Formula M3.

2.-6. (canceled)

7. The process according to claim 1, comprising
a) hydrogenating Formula M2 with 10% Pd/C and H₂ in NMP to provide Formula M3;
b) adding ACN to the solution of step (a); and
c) recovering Formula M3 as a crystalline form.

8. The process according to claim 1, comprising
a) hydrogenating Formula M2 with 10% Pd/C and H₂ in THE and H₂O to provide Formula M3;
b) adding ACN to the solution of step (a); and
c) recovering Formula M3 as a crystalline form.

9. An isolated crystalline form of N⁴-(3-methyl-4-[[1,2,4]triazolo[1,5-a]pyridin-7-yloxy]phenyl)quinazoline-4,6-diamine of Formula M3:

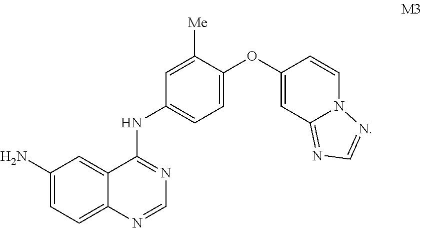 [SMILES: Cc1cc(Nc2ncnc3ccc(N)cc23)ccc1Oc1ccn2ncnc2c1]

10. The crystalline polymorph of claim 9, wherein the polymorph is Form A with X-Ray Diffraction Pattern (XRPD) peaks at 11.3, 11.5, 13.7, 21.5 and 26.0±0.2 degrees 2θ (CuKα).

11. (canceled)

12. The crystalline polymorph of claim 10, wherein the polymorph Form A has an XRPD pattern substantially the same as FIG. 1.

13. The crystalline polymorph of claim 9, wherein the polymorph is Form B with X-Ray Diffraction Pattern (XRPD) peaks at 11.9, 13.1, 15.9, 20.7, and 24.2±0.2 degrees 2θ (CuKα).

14. (canceled)

15. The crystalline polymorph of claim 13, wherein the polymorph Form B has an XRPD pattern substantially the same as FIG. 2.

16. The crystalline polymorph of claim 9, wherein the polymorph is Form C with X-Ray Diffraction Pattern (XRPD) peaks at 10.8, 13.1, 17.2, 24.9, and 27.2±0.2 degrees 2θ (CuKα).

17. (canceled)

18. The crystalline polymorph of claim 16, wherein the polymorph has an XRPD pattern substantially the same as FIG. 3.

19. An one-pot process for preparing the compound of Formula (I): 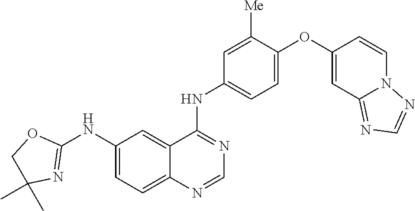 [SMILES: Cc1cc(Nc2ncnc3ccc(NC4=NC(C)(C)CO4)cc23)ccc1Oc1ccn2ncnc2c1]
or a pharmaceutically acceptable salt thereof, the process comprising:
a) reacting Formula M3: 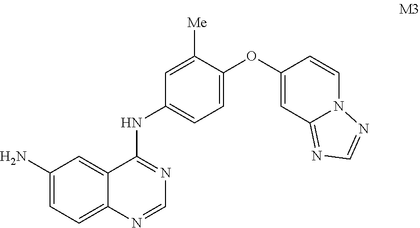 [SMILES: Cc1cc(Nc2ncnc3ccc(N)cc23)ccc1Oc1ccn2ncnc2c1]
with a coupling reagent and Formula SM3: 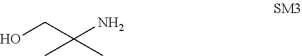
in a suitable solvent system to provide M4 reaction mixture, 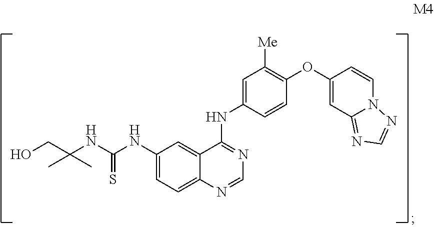
b) converting the M4 reaction mixture to Formula (I); and
c) optionally purify the Formula I.

20.-23. (canceled)

24. The process according to claim 19, wherein the step a) comprises reacting Formula M3 and the coupling reagent TCDI in DMF, to produce a M3IM reaction mixture:

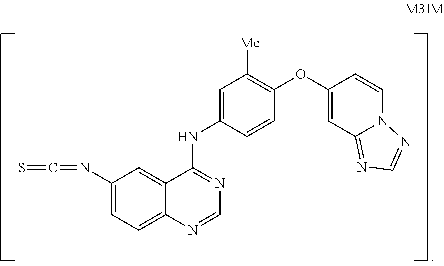

25. The process according to claim 24, wherein the step a) further comprises combining the M3IM reaction mixture and Formula SM3: 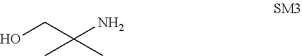
to produce the M4 reaction mixture.

26.-28. (canceled)

29. The process according to claim 19, wherein the Formula M3 is an isolated crystalline form of Formula M3 of claim 9.

30. The process according to claim 19, comprising
a) reacting Formula M3 with TCDI and Formula SM3 in DMF to provide the M4 reaction mixture; and
b) cyclizing the M4 reaction mixture to Formula (I) with air.

31. A process of preparing a compound of Formula (I) 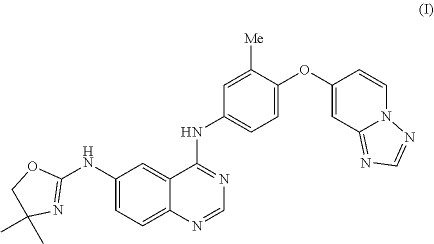 [SMILES: Cc1cc(Nc2ncnc3ccc(NC4=NC(C)(C)CO4)cc23)ccc1Oc1ccn2ncnc2c1]
or a pharmaceutically acceptable salt thereof, the process comprising:
adding a suitable cyclizing reagent to a M4 reaction mixture: 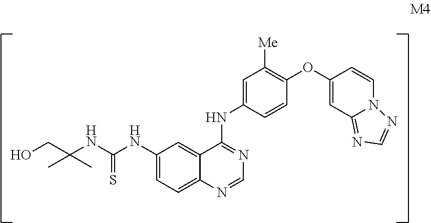
to prepare the compound of Formula I.

32. (canceled)

33. (canceled)

34. The process according to claim 31, wherein the M4 reaction mixture is prepared by reacting Formula M3: 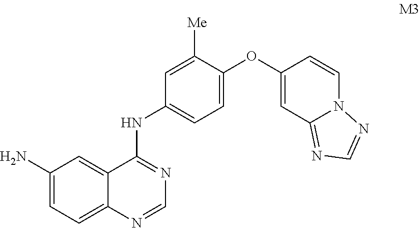 [SMILES: Cc1cc(Nc2ncnc3ccc(N)cc23)ccc1Oc1ccn2ncnc2c1]
with TCDI and Formula SM3: 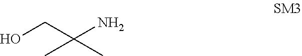
in DMF to provide the M4 reaction mixture.

35. The process according to claim 34, wherein the compound of Formula I is prepared by reacting Formula M3: 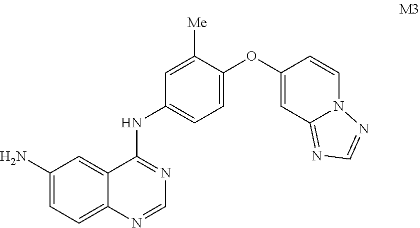 [SMILES: Cc1cc(Nc2ncnc3ccc(N)cc23)ccc1Oc1ccn2ncnc2c1]
with TCDI and Formula SM3: 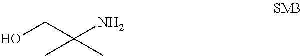
in DMF to provide the M4 reaction mixture; and
adding air to the M4 reaction mixture to prepare the compound of Formula I.

36. A process of preparing the compound of Formula (I) or a pharmaceutically acceptable salt thereof, the process comprising:
using a crystallized form of Formula M3 of claim 9 to perform a one-pot reaction of claim 19.

37. A process for preparing Tucatinib hemiethanolate, comprising dissolving Tucatinib API in a suitable organic solvent or a mixture of organic solvents and adding EtOH to prepare Tucatinib hemiethanolate.

38. (canceled)
